(19) United States
(12) Patent Application Publication
(10) Pub. No.: US 2017/0029762 A1
(43) Pub. Date: Feb. 2, 2017

(54) SEPARATION SYSTEM

(71) Applicants: PROMEDART BIYOTEKNOLOJI VE OZEL SAGLIK HIZMETLERI SANAYI TICARET LIMITED SIRKETI, Istanbul (TR); LITERAL MAKINE VE KIMYA SANAYI TICARET LIMITED SIRKETI, Istanbul (TR)

(72) Inventors: Gursel TURGUT, Istanbul (TR); Ahmet Reha BASARAN, Istanbul (TR); Ebru CATALTARLA SIRALI, Istanbul (TR); Kaya MOLO, Istanbul (TR)

(73) Assignees: PROMEDART BIYOTEKNOLOJI VE OZEL SAGLIK HIZMETLERI SANAYI TICARET LIMITED SIRKETI, Istanbul (TR); LITERAL MAKINE VE KIMYA SANAYI TICARET LIMITED SIRKETI, Istanbul (TR)

(21) Appl. No.: 15/295,180

(22) Filed: Oct. 17, 2016

Related U.S. Application Data
(63) Continuation of application No. PCT/TR2015/000152, filed on Apr. 14, 2015.

(30) Foreign Application Priority Data
Apr. 15, 2014 (TR) 2014/04356

(51) Int. Cl. C12M 1/00; B04B 5/04; A61M 1/36; C12M 1/26; C12M 1/34

(52) U.S. Cl. CPC C12M 47/02; C12M 33/10; C12M 41/46; A61M 1/3693; B04B 5/0442; A61M 1/3696; B04B 2005/0485

(57) ABSTRACT

This invention is about a separation system (1) that effectively separates the biological material into its different components with the effect of centrifugal force and performs processes such as separation of stem cells from bone marrow or fatty tissue with ease.

### TECHNICAL AREA

This invention is about a separation system that effectively separates the biological material into the different units in it with the help of centrifugal force.

### PREVIOUS TECHNIQUE

Centrifugation is a process which involves rotary motion on a fixed axis with the help of an electric motor. The high amount of revolutions per minute of the centrifuge makes the mixtures get separated based on principle of sedimentation. Based on their density differences, components accumulate toward the bottom part of the tube with the help of centrifugal force. Suspensions and emulsions could be easily separated in this manner. For example, blood can be separated into components in such a way that there is plasma on top, leukocytes in the middle and erythrocytes at the bottom.

In the current technique, different methods and equipment are used to process and separate the peripheral blood into its components. Sedimentation, one of the mentioned methods, is the process in which the cellular components which are homogenously dispersed in whole blood (the blood which has not been separated) get separated from the fluid as a consequence of gravity. Variational centrifugation is the process of collecting the needed cellular elements by increasing gravitational force by different amounts. As sedimentation is applied along with variational centrifugation, it becomes faster and more controllable with the addition of hydroxyethyl cellulose to whole blood.

In top and bottom technique, which is another method, every unit of blood that has gone through sedimentation is collected in different bags. A color monitored process is applied by the use of manual and optical systems.

In U.S. Pat. No 4,350,585 a blood separation equipment that separates the components of blood is described.

In another method that is currently used, the process of sedimentation and the collections of components in different bags is performed within a single system. In this system, blood is transferred to the sedimentation chamber via automation. Blood is separated into its components in this area by applying vertical centrifugation. Every component is collected by the help of color sensing optical controls. However, the results might turn out to be incomplete as the cell distinguishability is low in the systems used in -this technique. In addition, one cannot get the actual cell count from these type of equipment and the dead and living cells cannot be separated from each other.

### SHORT DESCRIPTION OF THE INVENTION

The invention concerns a device comprising a separation system that measures, evaluates and distinguishes the components which are simultaneously separated during or after separation.

It is a general object of the present invention to provide a separation system that weighs every byproduct that emerge as a result of the separation of the biological material using a load cell.

Another object of this invention is an improved separation system in which the volume of the whole blood can be measured with a contact free magnetic method.

Another object of this invention is to provide a separation system in which the speed of the piston and the shaft is adjustable by means of the air controlled system.

Another object of this invention is to provide a separation system wherein the selection of the components in low concentrations and counting of the cells can be easily conducted by means of speed regulation.

The size of the blood chamber defined in this inventions is modifiable horizontally and vertically. Accordingly, another object of this invention is to deliver a separation system which can automatically detect the size of the containers as they are placed into the system.

Another object of this invention is to provide a separation system wherein the living and dead cells can be separated from one another.

Another object of this invention is to deliver a separation system that separates the tissue into its components effectively by means of built-in color sensors.

Another object of this invention is to provide a separation system that has a modular assembly which helps acquire cells from bone marrow and fatty tissue besides blood.

Another object of this invention is to deliver a separation system that can process more than one blood sample simultaneously, thus is time- and space-saving.

### BRIEF DESCRIPTION OF THE FIGURES

The accompanying drawings illustrate a presently preferred embodiment of invention and together with the general description given above serve to explain the principles of the invention.

FIG. 1A is a first portion of a separation system embodying the present invention.

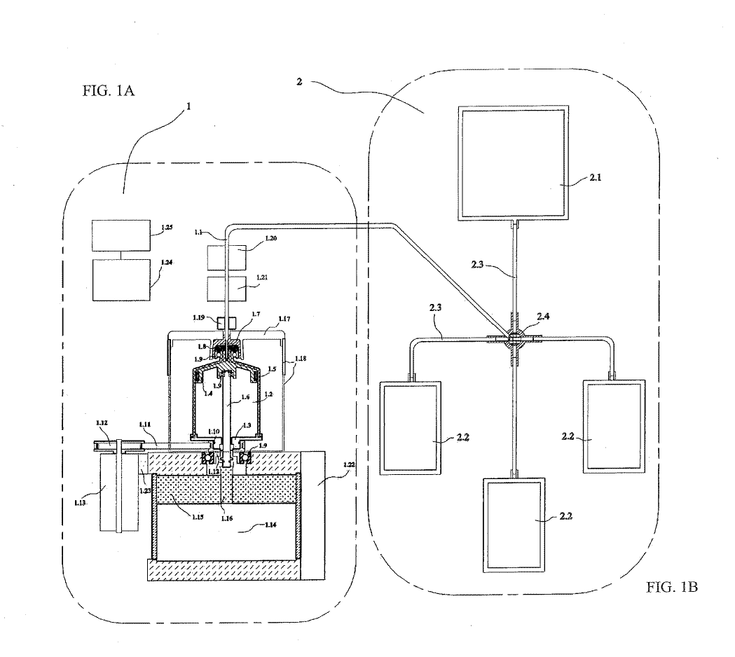

FIG. 1B is a second portion of a separation system embodying the present invention. FIG. 1C is a magnified view of the first portion illustrated in FIG. 1A.

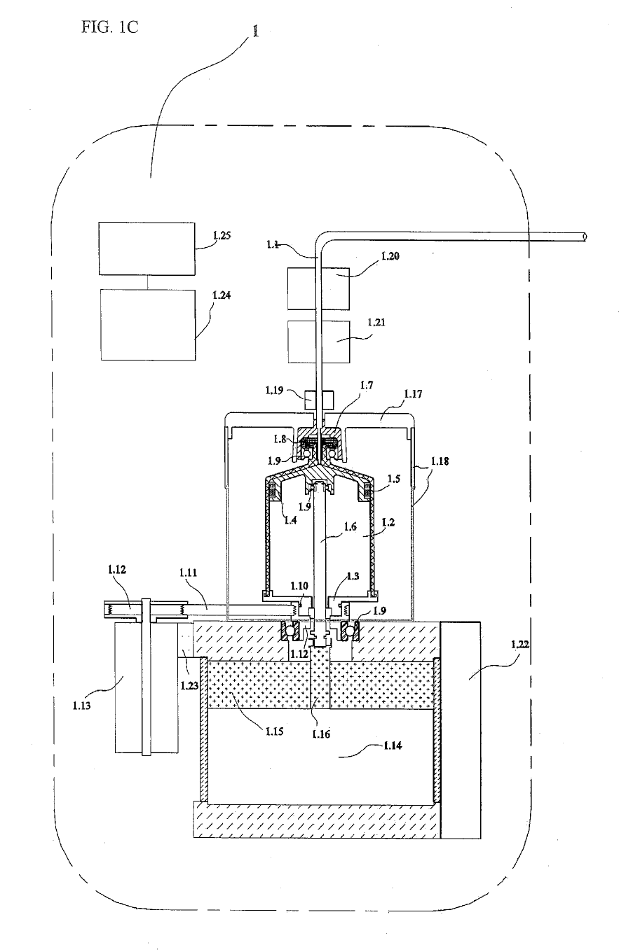

FIG. 1D is a magnified view of the second portion illustrated in FIG. 1B.

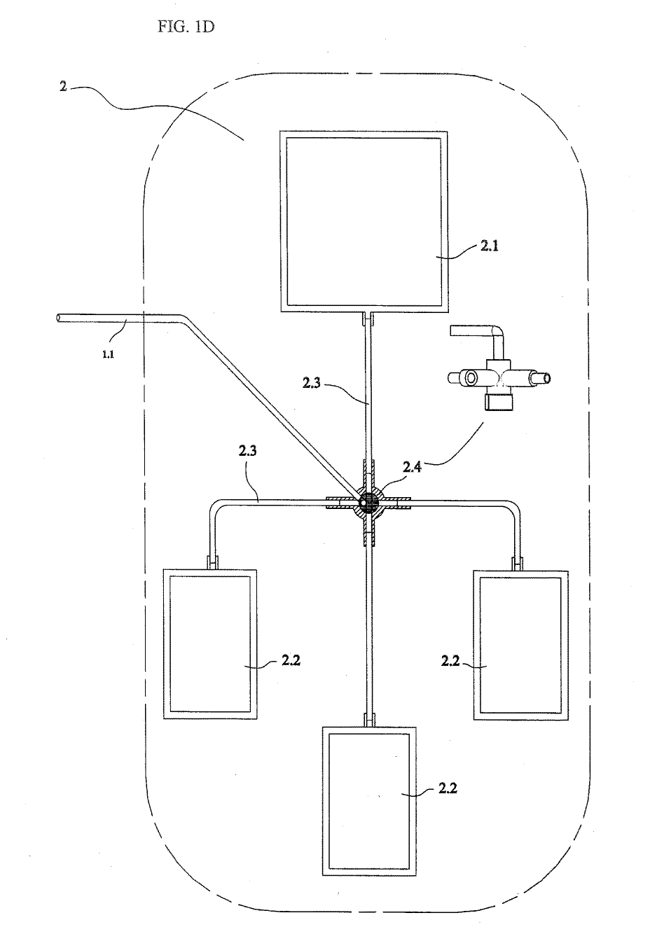

1. A separation system

1.1. transfer tube

1.2. separation chamber

1.3. Cover/lid

1.4. Piston

1.5. Silicone o-ring

1.6. piston shaft

1.7. Coupling

1.8. Teflon salmastra

1.9. Rulman

1.10. O-ring

1.11. Motor Kayis

1.12. Kasnak

1.13. Motor

1.14. cylinder chamber

1.15. compartment piston

1.16. Piston Lock

1.17. Chamber stabilizing cover

1.18. Device Body

1.19. Product identification sensor

1.20. Viability sensor

1.21. Cell counter

1.22. Position and volume reader

1.23. Shock absorber

1.24. Control Unit

1.25. Monitor

2. Kit

2.1. Processing chamber

2.2. product chamber

2.3. connector tubes

2.4. multiway valve

### DETAILED DESCRIPTION OF THE PREFERRED EMBODIMENTS

The separation system that separates and evaluates the biological material effectively into different components it contains by means of the centrifugal force (1) basically consists of
At least one kit in which the biological material is evaluated, distributed and preserved (2),
At least one chamber that can be plugged in and out, in which the biological material in the kit (2) is placed in a unseparated (whole) state (2.1)
At least one product chamber (2.2) in which the biological material can be preserved in separated state,
At least one connector tube (2.3) that transfers the whole biological material and the separated biological material from one place to another,
At least one multiway valve (2.4) that switches the course of the flow to the connector tubes (2.3) on or off as preferred,
At least one separation chamber (1.2) in which the biological material is placed and separated,
At least one transfer tube (1.1) that is placed between the kit (2) and the chamber (1.2) and transfers the whole biological material from the kit (2) to the chamber (2.1) and the separated biological material from the chamber (1.2) to the kit (2),
At least one piston (5) present within the processing chamber (1.2) and transfers the biological material from the initial container (2.1) to the separation chamber (1.2) by means of the vacuum present in the environment, that also transfers the components that are separated from the separation chamber (1.2) to the product container (2.2),by means of the pressure in the environment after the separation
At least one motor (1.13) on which the separation chamber (1.2) pivots to separate the biological material by means of centrifugal force,
At least one shock absorber (1.4) that absorbs and dissipates vibration of the motor (1.13) and ensures its quite operation,
At least one volume detector (1.23) within the chamber (1.2) that measures the volume of the biological material,
At least one product recognition sensor (1.19) on the transfer tube that identifies and differentiates the biological material that is placed into the chamber (1.2) and the separated biological material components which are transferred into the product container (2.2) and gives the movement commands of the multiway valve (2.4) based on the process,
At least one counting sensor (10) on the connector tube (2.3) that counts the cells,
At least one vitality sensor (1.20) on the connector tube (2.3) that determines the vitality of the cells,
At least one control unit (1.24) that compiles the information obtained from the vitality sensor (1.20), counting sensor (1.21), product sensor (1.19) and volume reader and controls the system accordingly.

In the said invention, the biological material is defined as at least one member or the combination of at least two members of the group which includes biological body fluid, cells, bone marrow and tissue such as blood, plasma, serum, infection, urine, saliva, semen. The biological material is preferably fluid in the usage of the invention.

In a preferred utilisation of the invention, a separation system (1) that separates the biological material into cells or different components that include more than one type of cell with the effect of centrifugal force is mentioned. The kit (2) that is used in a separation system (1) comprises a multiway valve (2.4), multiple connector tubes (2.3) connected to this multiway valve (2.4), product containers (2.2) at the end of these connector tubes (2.3) and an initial container (2.1). The initial container (2.1) is produced in four different sizes of 125, 250, 500 and 800 ml, preferably from plastic material that preferably varies from 100 ml to 1000 ml. The initial container (2.1) can be plugged on and off the transfer tube. The multiway valve (2.4) permits or prohibits the fluids to be transferred into the connector tubes (2.3) as preferred. The product containers (2.2) are used to collect the separated products. Different kits (2) are used for different kinds of separation processes. The kit (2) that is specifically designed for the preferred separation process is attached to a separation system (1) and which kit (2) is attached to which separation system (1) is defined.

A separation system (1) is comprised of a chamber (1.2). A least one leak proof lid (13) connects the kit (2) and the chamber (1.2). The biological material is transferred between the separation chamber (1.2) and the kit (2) through the transfer tube (1.1). Vacuum is generated in the separation chamber (1.2) when the piston (1.4) in the separation chamber (1.2) moves down. In addition, pressure is created in the separation chamber (1.2) when the piston (1.4) moves up. There is a compressed air-controlled compartment (1.14) under the separation chamber (1.2) that pushes and pulls the chamber piston (1.4). In this section, there is at least one air-controlled compartment piston (1.15).
The compressed air in the compartment (1.25) fills the compartment piston (1.15) and moves it. The compartment piston (1.15) moves up the piston shaft (1.6) and the piston (1.4) which are in the chamber (1.2) and are connected to the section piston (1.16) via the lock (1.16).
Vacuum generated in this section (1.25) moves the compartment piston (1.15) by withdrawing the air in the cylinder chamber (1.14). The compartment piston (1.15) moves and the piston (1.4) and the piston shaft in the chamber down (1.5) (1.2) and are connected to the chamber piston (1.16) via the lock.

A volume detector (1.22) that is outside the compressed air and the vacuum-controlled chamber (1.14 magnetically) measures the volume of the fluid that is in the separation chamber (1.2). The volume of the separation chamber (1.2) used and the analog input signal generated by the volume detector (1.23) analysed by the control unit (1.24) are used to determine the volume processed fluid.

The motor (1.13) outside the separation chamber (1.2) preferably runs at 0-20.000 revolutions per minute, and revolves the chamber (1.2) at this speed. Centrifugal force is generated and the biological material is separated at the preferred amount as a result of the motor (1.13) revolving the separation chamber (1.2).

To prevent the engine (1.13) from creating noise and vibration when running at a high cycle, the motor (1.13) is fastened to the cylinder's (1.14) body with the shock absorber (1.23).

The product sensor (1.19) determines the biological material transferred to the separation chamber (1.2) and to which final product container (2.2) the separated components should be transferred. Once separated, all the components of the biological material can be distinguished by the optical differences in their color. The sensor (1.19) recognizes the changes in the color, hence, the related product, and signals the controller to direct the product to its final chamber (2.2) by controlling the movement of the multiway valve (2.4).

The invention is further described by the following nonlimiting example:

Blood, tissue or biological fluid to be separated is placed in the initial container (2.1) of the kit (2). The multiway valve (2.4) opens the path between the initial container (2.21) and the separation chamber (1.2) on and closes the paths to the product containers (2.2). The piston (5) in the separation chamber (1.2) moves down and vacuum is created in the separation chamber (1.2). As a result, blood, tissue or biological fluid is transferred from the initial container (2.1) to the separation chamber (1.2) via connector tubes (2.3) and the transfer tube (1.1). Volume detector (1.23) determines the volume of the fluid that is in the separation chamber (1.2) and controls the preferred amount of biological material be placed into the separation chamber (1.2) as the biological material is transferred into the separation chamber (1.2). Once the preferred amount of fluid is transferred into the separation chamber (1.2), the motor (1.13) is activated and starts rotating the chamber (1.2)

The separation chamber (1.2) is rotated based on the preferred amount and duration. Rotation of the chamber (1.2) causes the particles to move outward in the radial direction with respect to their densities. Later on, the piston (1.4) moves up and creates pressure in the separation chamber (1.2) which transfers the fluids in the direction of the transfer tube (1.1). Whether the fluid that is on the top enters the transfer pipe (1.1) or not is determined by the product sensor (1.19) and a volume detector (1.22) determines the volume of the fluid in each layer. The multiway valve (2.4) opens or closes the paths to relative the transfer tubes so that the fluid could be directed to different product containers (2.2). Thus, all fluids can be placed in the preferred product container (2.2) separately.

There is at least one counting sensor (1.21) on the connector tubes (2.3) and counts the cells before the components of the separated material is transferred to the product containers (2.2). Counting sensor (1.21) counts the living and dead cells until the product sensor (1.19) identifies the next separated product.

There is at least one vitality sensor (1.20) that is on the connector tubes (2.3) and determines the vitality of the cells before the separated biological material component is transferred into the product chamber (2.2). The vitality sensor (1.21) creates an analog signal by identifying the living cells that are in the products that are transferred to the product chambers. Amount of the living cells is determined by evaluation of the signal at the control unit (1.24.

The control unit (1.24) is responsible for the on and off direction of the multiway valve (2.4) by using the information received from the sensors (1.19, 1.20, 1.21) and the reader (1.23). The control unit also calculates the volume and the cell amount by evaluating the analog data submitted by the sensors (1.19, 1.20, 1.21) and the reader (1.23), and shows the information on the monitor, plasma separation, stem cell separation from bone marrow or fatty tissue can easily be performed by using a separation system (1) in addition to separation of blood, tissue or biological fluids.

Additional advantages and modifications will readily occur to those skilled in the art. Therefore, the invention is not limited to specific details and illustrative examples shown and described herein. Accordingly, this inventions is to be limited only by the scope of the appended claims.

1. A separation system (1) which separates and evaluates biological material into different components by means of centrifugal force, and also is basically characterized by,
At least one kit (2) in which the biological material is evaluated, distributed and preserved,
At least one separation chamber (1.2) in which the biological material is placed and separated,
At least one transfer tube (1.1) that is between the kit (2) and the chamber (1.2) and transfers the raw biological material from the kit (2) to the chamber (1.2) and the separated biological material from the container (1.2) to the kit (2),
At least one piston (1.4) that is in the chamber (1.2) and transfers the biological material from the initial container (2.1) into the container (1.2) by means of vacuum and pressure created in the environment, that also transfers the separated components from the container (1.2) to the product container (2.2) by creating pressure in the environment,
At least one motor (1.13) on which the chamber (1.2) pivots in order to separate the biological material by means of centrifugal force,
At least one shock absorber (1.23) that absorbs and dissipates the vibration of the motor (1.13) and ensures its quite operation,
At least one volume detector (1.22) that is in the chamber (1.2) and determines the volume of the biological material,
At least one product sensor (1.19) that is on the transfer tube (1.1) and identifies and differentiates between the biological material that is transferred into the chamber (1.2) and the separated biological material components which are transferred into the product chamber (2.2) and sends the movement commands relating to the process,
At least one counting sensor (1.21) that is on the connector tube (2.3) and counts the cells,
At least one vitality sensor (1.20) that is on the connector tube (2.3) and determines the vitality of the cells,
At least one control unit (1.24) that compiles the information obtained from the vitality sensor (1.20), counting sensor (1.21), product sensor (1.19) and volume reader (1.22) and checks the system accordingly.

2. A separation system (1) as claimed as in claim 1, comprising at least one initial container (2.1) ton the kit (2) that can be plugged in and out, in which the biological material is placed in a unseparated state (2.1).

3. A separation system (1) as claimed in claim 1, comprising—at least one product chamber (2.2) in which the separated biological material can be preserved after the separation process.

4. A separation system (1) is claimed in claim 1, further comprising at least one connector tube (2.3) that transfers the raw biological material and the separated biological material from one container to another.

5. A separation system (1) as claimed in claim 1, further comprising at least one multiway valve (2.4) that switches the course to the connector tubes (2.3) on or off as preferred.

6. A separation system (1) as claimed in claim 1, further comprising at least one leak proof lid (13) that connects the kit (2) and the separation chamber (1.2).

7. A separation system (1) as claimed in claim 1, further comprising at least one shock absorber (7) that equilibrates the motor (6) and ensures its quite operation

8. A separation system (1) as claimed in claim 1, comprising the kit (2) which includes a multiway valve (2.4) and multiple connector tubes (2.3) connected to the multiway valve (2.4), product containers (2.2) at the end of these connector tubes (2.3), and an initial container (2.1).

9. A separation system (1) as claimed in claim 1, which is characterized by the multipath valve (2.4) which allows or stops the fluids that are in the kit (2) to be transferred/from being transferred into the connector tubes (2.3) with the help of the control unit.

10. A separation system (1) as claimed in claim 1, which is characterized by the product chamber (2.2) that is in the kit and in which the separated product is accumulated.

11. A separation system (1) as claimed in claim 1, which is characterized by the piston (1.4) that is in the chamber (1.2) and creates vacuum within the chamber (1.2) if it moves down and creates pressure in the chamber (1.2) if it moves up.

12. A separation system (1) as claimed in claim 1, which is characterized by the engine (1.13) that is under the chamber (1.2) and preferably runs at 0-20.000 revolutions per minute, and revolves the chamber (1.2) at this speed, creating centrifugal force as a result of revolving the chamber (1.2).

13. A separation system (1) as claimed in claim 1, which is characterized by biological material that is a member or the combination of at least two members of the group which includes biological body fluids, cells, bone marrow and tissue such as blood, plasma, serum, pus, urine, saliva, semen.
